(19) United States
(12) Patent Application Publication
(10) Pub. No.: US 2023/0240842 A1
(43) Pub. Date: Aug. 3, 2023

(54) Flexible Heart Valve with Arm Attachment to Outside Structure

(71) Applicant: Cephea Valve Technologies, Inc., Abbott Park, IL (US)

(72) Inventors: Grayston Licht, Riverside, CA (US); Randolf Von Oepen, Aptos, CA (US)

(73) Assignee: Cephea Valve Technologies, Inc., Abbott Park, IL (US)

(21) Appl. No.: 18/060,644

(22) Filed: Dec. 1, 2022

Related U.S. Application Data
(60) Provisional application No. 63/304,830, filed on Jan. 31, 2022.

(51) Int. Cl. A61F 2/24

(52) U.S. Cl. CPC A61F 2/2418; A61F 2220/0041; A61F 2220/0091; A61F 2230/0026

(57) ABSTRACT

The present disclosure provides replacement heart valves that include an outer frame, an inner frame, and arms extending therebetween. The outer frame and inner frame may be formed by a plurality of cells, such as diamond shaped cells. The arms may extend from an apex of a diamond shaped cell on the inner frame and be fastened to an apex where adjacent cells of the outer frame adjoin. The arms may include one or more bends. For example, the arms may extend in a first direction for a predefined length, have a bend point, and then extend in a second direction opposite the first. The arms may isolate the inner frame from the outer frame such that forces exerted on the outer frame are absorbed by the arms and do not deform the inner frame.

### CROSS-REFERENCE TO RELATED APPLICATIONS

This application claims priority to the filing date of U.S. Provisional Patent Application No. 63/304,830, filed Jan. 31, 2022, the disclosure of which is hereby incorporated by reference herein.

### BACKGROUND OF THE DISCLOSURE

Replacement heart valves may include two frames, such as an outer frame and an inner frame. The outer frame is used to anchor the structure while the inner frame is used to hold the leaflets. The size of the inner frame may relate, or correspond, to the effective orifice area (“EOA”). The EOA and, therefore, the size of the inner frame often remains constant even when the annulus is dilated to a different size. For example, a replacement mitral valve can have an outer frame between 32 mm and 48 mm. The inner frame and, therefore, the valve, is to be kept at a constant size, such as 27 mm or 29 mm, regardless of the size of the outer frame. The larger the size of the inner frame the lower the pressure gradient should be.

A larger size inner frame provides a greater possibility to perform a valve in valve procedure when the replacement heart valve begins to deteriorate. A larger inner frame and, therefore, larger valve size, results in a greater amount of stress on the leaflets as compared to a smaller inner frame and valve. Physicians expect that a valve in valve procedure has to be performed at least once and as many as three times, if not more. Therefore, it is necessary to balance valve leaflet durability, EOA, and the ability to perform valve in valve procedures.

The outer and inner frames of the replacement valve may be rigid and/or manufactured as a single structure. This may prevent the physician from being able to select a combination of sizes of an outer frame and an inner frame. Moreover, with a rigid outer and inner frame, the inner frame may be deformed by any force exerted on the external frame. Thus, the inner frame may not maintain its shape.

### BRIEF SUMMARY

The disclosure is generally directed to a replacement heart valve having an outer frame, an inner frame, and a plurality of arms therebetween. The outer frame may be configured to engage the dilated heart annular area and the inner frame may be configured to carry the heart valve tissue and sealing fabrics. Each of the outer frame and inner frame may include a plurality of cells. In some examples, the cells may be diamond shaped cells. The arms may extend from the inner frame and fasten to the outer frame, such that the arms couple, or connect, the outer and inner frame. According to some examples, the arms may extend from the apex of a cell of the inner frame and be coupled to an apex where adjacent cells of the outer frame adjoin. In some examples, the arms may have more than one portion such that a first portion of the arm extends in a first direction and a second section of the arm extends in a second direction opposite the first with a hinge, or a bend, between the first section and the section portion. For example, a first section of the arm may extend from an apex of a cell on the atrial end of the inner frame towards the ventricular end, a second section of the arm may extend back towards the atrial end, and there is a hinge, or a bend point between the first section and the second section such that the arms form a “zig-zag” shape. In some examples, there may be more than one bend point, such that the arms form a “zig-zag-zig” shape. In other examples, there may not be a bend in the arm, such that the arms are straight. The length of the arm, the number of bend points, the thickness of the material forming the arm, etc. may determine how much force the arms can absorb. For example, an increase in the number of bend points in each of the arms may increase the independence of the inner frame from the outer frame. Independence of the inner frame from the outer frame may prevent the inner frame from deformation such that the valve remains circular even when the outer frame is deformed.

The arms may absorb forces exerted on the outer frame to prevent or alleviate the force from being exerted on the inner frame. That is, the arms may isolate the inner frame such that a force exerted on the outer frame is absorbed (including being partially absorbed) by the outer frame and/or arms rather than the inner frame. This may allow for the inner frame to maintain its circular shape (or ovalize less) regardless of the force exerted on the outer frame of the replacement heart valve. In some examples, the arms may allow for both lateral and axial movement of the inner frame during a heart cycle to be isolated.

One aspect of the disclosure is directed to a replacement cardiac valve, comprising an outer frame configured to expand from a collapsed configuration to an expanded configuration, an inner frame configured to expand from a collapsed configuration to an expanded configuration, and one or more arms extending between the inner frame and the outer frame, wherein the one or more arms are configured to isolate the inner frame such that a force exerted on the outer frame is absorbed (including being partially absorbed) by the outer frame and the one or more arms.

The one or more arms may extend from the inner frame and may be coupled to the outer frame. The outer frame may include a first plurality of diamond shaped cells and the inner frame may include a second plurality of diamond shaped cells such that each of the one or more arms extends from an apex of the second plurality of cells to a connection point between adjacent cells of the first plurality of cells. Each of the one or more arms may be coupled to the outer frame by a rivet, a suture, a polymer, or an interlocking system.

The one or more arms may have at least one hinge point. Each of the at least one hinge points may correspond to a change in direction of the arms. The one or more arms may be a tether. A circumference of the outer frame may include a first number of cells and a circumference of the inner frame may include a second number of cells. The first number of cells may be a multiple (i.e. a whole-number multiple) of the second number of cells.

Another aspect of the disclosure is a replacement cardiac valve comprising an outer frame having a first plurality of diamond shaped cells, the outer frame configured to expand from a collapsed configuration to an expanded configuration, an inner frame having a second plurality of diamond shaped cells, the inner frame configured to expand from a collapsed configuration to an expanded configuration, and one or more arms extending from the inner frame to the outer frame, wherein each of the one or more arms extends from an apex of the second plurality of diamond shapes cells and is coupled to an apex of the first plurality of diamond shaped cells.

Yet another aspect of the disclosure is a replacement cardiac valve comprising an outer frame configured to expand from a collapsed configuration to an expanded configuration, an inner frame configured to expand from a collapsed configuration to an expanded configuration, and one or more arms extending from the inner frame and coupled to the outer frame, wherein each of the one or more arms has a bend point corresponding to a change in direction of each of the one or more anchoring mechanisms, and wherein the one or more arms are configured to isolate the inner frame such that a force exerted on the outer frame is absorbed (including being partially absorbed) by the outer frame and the one or more arms.

Yet another aspect of the disclosure is a replacement valve comprising an outer frame having a first plurality of diamond shaped cells, the outer frame configured to expand from a collapsed configuration to an expanded configuration, an inner frame having a second plurality of diamond shaped cells, the inner frame configured to expand from a collapsed configuration to an expanded configuration, and one or more arms extending from an apex of the second plurality of cells to a connection point between adjacent cells of the first plurality of cells, wherein each of the one or more arms includes one or more portions, a first portion of each of the one or more arms extending in a first direction and a second portion of each of the one or more arms extending in a second direction opposite the first direction, wherein each of the one or more arms has a bend point between the first portion and the second portion, and wherein the one or more arms are configured to isolate the inner frame such that a force exerted on the outer frame is absorbed by the outer frame and the one or more arms.

### BRIEF DESCRIPTION OF THE DRAWINGS

FIG. 1 is a perspective view of an outer frame of a replacement heart valve in an expanded configuration according to aspects of the disclosure.

FIGS. 2A-2C are perspective views of various inner frames of a replacement heart valve in an expanded configuration according to aspects of the disclosure.

FIG. 3 is a perspective view of the connection between the inner frame and outer frame of a replacement heart valve according to aspects of the disclosure.

FIG. 4 is a perspective view of a replacement heart valve according to aspects of the disclosure.

### DETAILED DESCRIPTION

Replacement heart valves may use a combination of outer frames and inner frames, each having different diameters. The outer frame may be configured to anchor the replacement heart valve in the natural annulus and the inner frame maybe configured to include the heart valve and sealing tissue. It would be beneficial for the outer frame to deform independently from the inner frame such that the valve connected to the inner frame remains as close to circular as possible during the deformation of the outer frame.

The inner and outer frames may be coupled via one or more arms extending from the inner frame and being coupled to the outer frame. The arms may be of various shapes and lengths. For example, the arms may have multiple sections such that a first section extends in a first direction and a second section extends in a second direction with a bend point between the first and second sections. Thus, the arm may have a shape like a check-mark. In some examples, the arms may have three portions, such that the arms have a zig-zag-zig shape.

The arms may be coupled to the outer frame via rivets, sutures, polymer, etc. For example, there may be a connection point on the outer frame and a connection point on the end of the arm. The connection point on the end of the arm may align with the connection point on the outer frame when the inner frame is within a central opening of the outer frame. According to some examples, the outer and inner frames may include a plurality of cells, such as diamond shaped cells. The arm may extend from an apex of a diamond shaped cell of the inner frame and be coupled to a connection point on the outer frame where two adjacent cells adjoin.

The one or more arms may allow the inner frame, and therefore the valve mounted therein, to be independent and flexible in movement from the outer frame. This may isolate the inner frame from any outer annular movement from the valve pulsatile loads and motion. For example, a force exerted on the outer frame may be transferred to and/or absorbed (including being partially absorbed) by the arms such that the inner frame does not experience the force (or the entire force). Additionally or alternatively, the arms may allow for lateral and/or axial movements in the heart cycle to be isolated from the inner frame and the valve mounted therein. For example, the mitral valve is a part of the ventricular structure having a D-shape and/or a saddle shape. The replacement valve may have a round, or circular, shape that should remain as close to circular as possible during the cardiac cycle even though the native valve changes shape during the cardiac cycle. By isolating the inner frame from the outer frame via one or more arms, the outer frame may be deformed to the shape of the natural valve during the cardiac cycle while the inner frame, and therefore the replacement valve does not become deformed. According to some examples, the one or more arms may absorb (including partially absorb) the lateral force exerted on the outer frame such that the inner frame may deform less as compared to replacement valves without the one or more arms.

According to some examples, the one or more arms may allow the inner frame to remain constant regardless of the frame that is chosen. For example, the inner frame may be a predetermined size, such as 27 mm, 29 mm, 33 mm inner frame. The outer frame may be any size. In some examples, the outer frame may have an outer diameter of between about 36 mm to 60 mm at the central waist portion. The one or more arms may be stretched between the inner frame and the outer frame, allowing various combinations of an inner frame and an outer frame. This may allow for a valve and, therefore, an inner frame that is typically used for a first valve replacement to be used in a second valve location as the outer frame may be selected based on the location of the valve replacement, rather than the chosen valve and/or inner frame. For example, an inner frame and valve intended for an aortic valve replacement may be used with no modifications (or only slight modifications) for a triscupid valve replacement as an appropriate outer frame may be selected and coupled to the inner frame via the one or more arms.

The replacement heart valve, including the outer frame and inner frame, described herein, may be used as a replacement aortic, pulmonary, mitral, or tricuspid valve. When describing certain features, reference is made to orientations of the replacement heart valve based on use as a mitral valve. For example, the disclosure herein may refer to an atrial end and a ventricular end. This is for reference and ease of understanding and is not intended to be limiting to use as a replacement mitral valve.

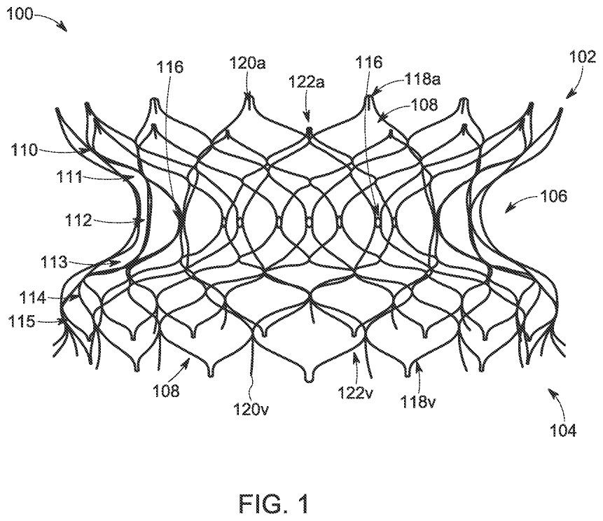

FIG. 1 is a perspective view of an outer frame of a replacement heart valve in an expanded configuration. The outer frame 100 may be configured to engage the dilated heart annular area. The outer frame 100 may have an atrial end 102, ventricular end 104, and central portion 106 therebetween. The atrial end 102 may be configured and adapted to be disposed on an atrial side of a mitral valve orifice and ventricular end 104 may be configured and adapted to be disposed on a ventricle side of the mitral valve orifice. In some examples, however, the outer frame 100 may be implanted so that atrial end 102 is positioned on the ventricle side and the ventricular end 104 is positioned on the atrial side. The atrial end 102 and ventricular end 104 may extend radially outward from ventral portion 106. In some examples, the atrial end 102 and ventricular end 104 may flare outward relative to central portion 106. Additionally or alternatively, atrial end 102 and ventricular end 104 may be flanged or flared relative to central portion 106.

The outer frame 100 may be cut (e.g., laser cut) out of a flat sheet of material or a tube. In some examples the material is a nitinol sheet or tube and shape set according to a valve position and/or anatomic design. For example, the outer frame 100 may be shape set for use in an aortic, pulmonary, mitral, or tricuspid valve. According to some examples, the nitinol sheet is 0.1 mm to 1.0 mm thick. In other examples, the nitinol sheet and/or tube may be 0.2 mm to 0.6 mm thick. However, the ranges of the thickness of the nitinol sheet and/or tube are merely examples and are not intended to be limiting.

Outer frame 100 may comprise a plurality of diamond shaped cells 108. According to some examples, the plurality of diamond shaped cells 108 may create rows of cells 108. As shown, there may be five rows of cells. For example, the atrial end 102 may be a first row 110 of cells, between the atrial end 102 and the central portion 106 may be a second row 111 of cells, the central portion 106 may be a third row 112 of cells, between the central portion 106. The ventricular end 104 may have a fourth row 113 of cells, fifth row 114 of cells, and a sixth row 115 of cells. According to some examples, the third row 112 of cells may have fewer or more cells than the first row 110 or sixth row 115 of diamond shaped cells. While outer frame 100 is shown with six rows of cells 108, there may be any number of rows based on the size, shape, configuration, etc. of cells. According to some examples, outer frame 100 may not include rows of cells 108 due to the shape and/or configuration of the cells.

The diamond shaped cells 108 may have configurations other than general diamond shapes, such as hexagonal, triangle, etc. In some examples, the diamond shaped cells 108 may not all have the same configuration. For example, each row 110-115 may have a different shape. In some examples, the first row 110, third row 112, and sixth row 115 of cells may be a first shape and the second row 111, fourth row 113, and fifth row 114 may be a second shape. However, there may be any combination of shapes and numbers of cells among the first, second, third, fourth, fifth, and sixth rows 110-115 of cells.

As shown in FIG. 1, between each diamond shaped cell 108 in the third row 112 of cells, there may be an aperture 116. The aperture 116 may be formed where adjacent cells 108 in the second row 112 of cells meet or adjoin. The apertures 116 may be aligned axially (i.e., as shown they are in a “row”), but in other examples they may not be aligned axially. For example, if the cells 108 have different configurations, some apertures 116 may not be aligned, or in the same “row” with other apertures. In some examples, the apertures 116 may be located where the cells 108 in the third row 112 of cells meet the cells 108 of the second row 111 or fourth row 113 of cells. Thus, while the apertures 116 are shown as being aligned axially where adjacent cells 108 in the third row 112 of cells meet, the apertures 116 may be located at the apex of the cells 108 in the third row 112 of cells. According to some examples, the apertures may, additionally or alternatively, be located where adjacent cells 108 meet in the second row 111 or fourth row 114 and/or at the apex of the cells 108 in the second row or fourth row 114. Thus, having the apertures 116 aligned axially in the third row 113 of cells is merely one example of the location of the apertures 116 and is not intended to be limiting.

Outer frame 100 may include a plurality of arches 118v that extend from the central portion 106 to the ventricular end 104 and a plurality of arches 118a that extend from the central portion 106 to the atrial end 102. The configuration of arches 118a, 118v are generally triangular-shaped, “pointing” towards the atrial end 102 or ventricular end 104, and include two sections of material and a bend between the two sections of material. As shown, in each arch 118a, 118v, the material first extends away from central portion 106, forms a bend at the atrial end 102 or ventricular end 104, respectively, of outer frame 100, and then extends back towards central portion 106. Arches 118a, 118v are triangular shaped in this example, and the ventricular ends can be described as tips of the arches. In some examples, the tips of the arches are rounded. The tips at the ventricular end 104 may be rounded, or curved, to avoid damaging the tissue when implanted. However, in some examples, the tips of the arches may be pointed. Additionally or alternatively, the tips of the arches may include an aperture or hook. For example, the tips of arches 118 may include a pin 120a extending from the tip. The pins 120a may face in towards the central opening of outer frame 100. The pins 120a may be used to connect the replacement valve to a delivery system. In some examples, the replacement valve may be connected to the delivery system via sutures to resist pressure build-up.

A plurality of spaces 122a, 122v may be positioned between between adjacent arches 118a, 118v. The configurations and sizes of the spaces 122a, 122v may be defined by the configuration of adjacent arches 118a, 118v. In some embodiments, the spaces 122v may be configured to allow the sub-valvular structures, such as chords, to slide between adjacent arches 118v when the outer frame is expanded. However, if sealing member (e.g. an inner and/or outer skirt) is provided on the outer frame 100, and if that sealing member extends all the way down to the arches 118v, that sealing member may occupy the spaces 122v, in which case those spaces 122v may not be able to receive any portion of the sub-valvular structure therein. The configuration of the arches 118a, 118v, and thus the plurality of spaces 122a, 122v, may provide easier collapse of the outer frame 100 radially inwards when the anchor is collapsed (e.g., for delivery).

According to some examples, a plurality of hooks may extend from where adjacent cells 108 meet in the spaces 122a, 122v. The hooks 120v may help anchor the replacement valve into the tissue. As shown, where adjacent cells 108 of the sixth row 115 of cells meet, hooks 120v may extend, or flare, outwardly. While the hooks 120v are shown as being located where adjacent cells 108 of the sixth row 115 of cells meet, hooks 120v may be positioned at various locations on outer frame 100. For example, hooks 120v may be positioned at the proximal end of the fifth row of cells 114. In some examples, hooks 120v may be positioned at the proximal end of the cells if any row and/or in combination with hooks 120v on multiple rows.

Although not shown in FIG. 1, it should be understood that the outer frame 100 may include one or more sealing members, such as a skirts, on the inner and/or exterior surface of the outer frame to assist with sealing against the anatomy, mitigating or preventing leakage through or around the outer frame 100, and/or enhancing eventual tissue in-growth into the prosthetic heart valve.

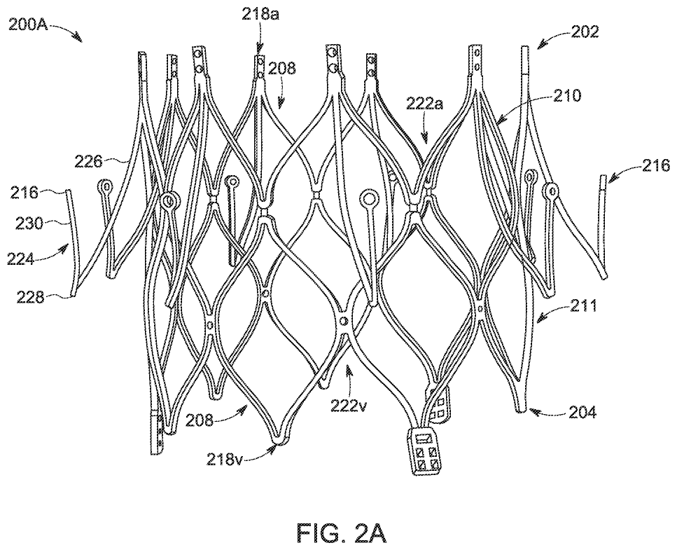

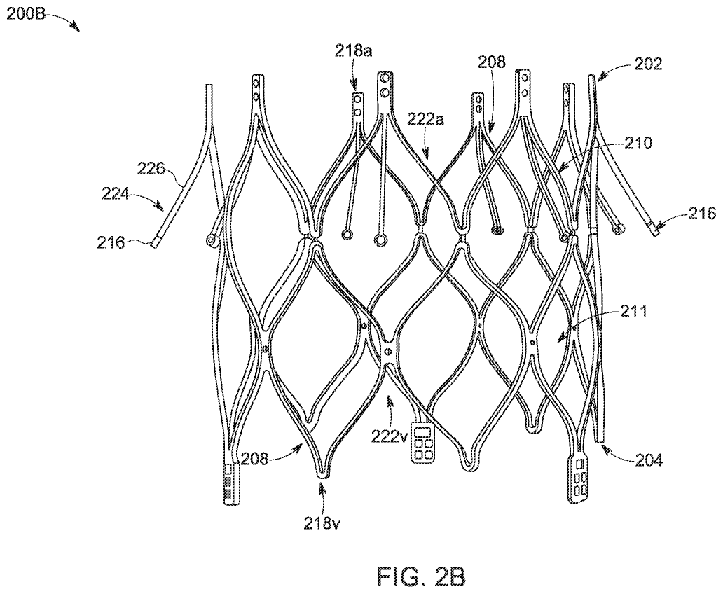

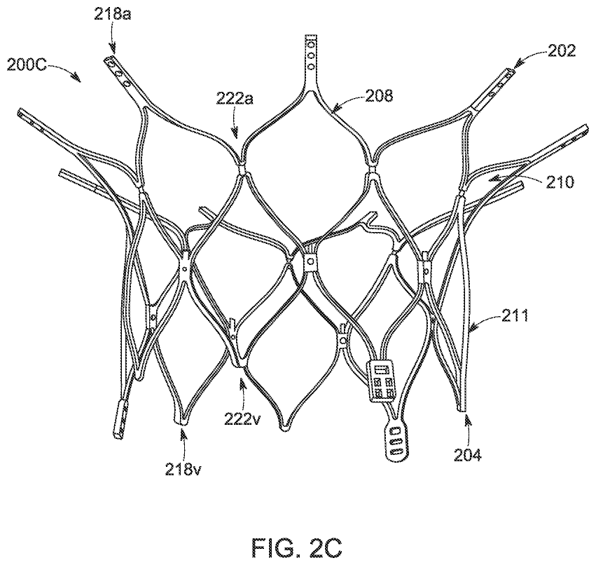

FIGS. 2A-2C are perspective views of example inner frames of a replacement heart valve in an expanded configuration. Each inner frame 200A, 200B, 200C may have a similar configuration and, therefore, similar reference numbers may be used. While the disclosure provided herein may refer to inner frame 200A, the disclosure also applies to inner frames 200B, 200C.

The inner frame 200 may be used for different applications, such as for a mitral valve, tricuspid valve, aortic valve, pulmonary valve, etc. The inner frame 200 may carry the heart valve tissue (e.g. a plurality of tissue or synthetic leaflets that close and open to provide the valve functionality) and one or more sealing members (e.g. tissue or fabric on the inner and/or outer surface of the inner frame 200 and/or tissue or fabric extending between the inner frame 200 and the outer frame 100 to at least partially seal the space between the inner frame 200 and outer frame 100). The inner frame 200 may have an atrial end 202 and a ventricular end 204. The atrial end 202 may be configured and adapted to be disposed on an atrial side of a mitral valve orifice and ventricular end 204 may be configured and adapted to be disposed on a ventricle side of the mitral valve orifice. In some examples, however, the inner frame 200 may be implanted so that atrial end 202 is positioned on the ventricle side and the ventricular end 104 is positioned on the atrial side.

The inner frame 200 may be cut (e.g., laser cut) out of a flat sheet of material or a tube. In some examples the material is a nitinol sheet or tube and shape set according to a valve position and/or anatomic design. According to some examples, the nitinol sheet is 0.1 mm to 1.0 mm thick. In other examples, the nitinol sheet and/or tube may be 0.2 mm to 0.6 mm thick. However, the ranges of the thickness of the nitinol sheet and/or tube are merely examples and are not intended to be limiting.

Inner frame 200 may comprise a plurality of diamond shaped cells 208. According to some examples, the plurality of cells may create rows of cells 208. As shown, there may be two rows of cells. For example, the atrial end 202 may be a first row 210 of cells and the ventricular end 204 may be a second row 211 of cells. While only two rows 210, 211 of cells are shown, there may be any number of rows of cells in the inner frame.

The diamond shaped cells 208 may have configurations other than general diamond shapes, such as hexagonal, triangle, etc. In some examples, the diamond shaped cells 108 may not all have the same configuration. For example, each row 210, 211 may have a different shape.

Inner frame 200 may include a plurality of arches 218a that extend from where the first row 210 of cells and the second row of cells 211 meet towards the atrial end 202 and a plurality of arches 218v that extend from where the first row 210 of cells and the second row of cells 211 meet towards the ventricular end 204. The arches 218a, 218v may be similar in shape and/or configuration as arches 118a, 118v. Between adjacent arches 218a, 218v may be a plurality of spaces 222a, 222v, similar to spaces 122a, 122v.

A plurality of arms 224 may extend from the tips of arches 218a. According to some examples, the arms may additionally or alternatively be referred to as extension members, tethers, etc. The arms 224 may extend from the tip of arch 218a towards ventricular end 204. During manufacturing of the inner frame 200, the arms 224 may be nested inside of cells 208.

As shown in FIG. 2A, arms 224 may include two sections, or portions, of material and a bend between the two sections. For example, the first section 226 of material may extend from arch 218a towards the ventricular end 204, form a bend 228, and then the second section 230 of material extends back towards the atrial end 202. The first section 226 may extend from the tip of arch 218a or a portion of arch 218a substantially near the tip of arch 218a. The first section 226 of material may extend beyond the height of cell 208, substantially the height of cell 208, or less than the height of cell 208 before forming bend 228 and extending back towards the atrial end 202. The height of the cell may be the distance from the tip of arch 218a to the opposing apex of the diamond shape cell 208. The length of the first section 226 of material may be varied based on the desired stiffness of arm 224. The stiffness of arms 224 may correspond to the amount of force the arms 224 can absorb. Increasing the length of arm 224 may increase the amount of force that may be absorbed by the arms 224. Increasing the amount of force absorbed by arms 224 may increase the amount of isolation of the inner frame 200 from outer frame 100. Increasing the amount of isolation of inner frame 200 may prevent inner frame 200 from being deformed (or at least minimize the amount that the inner frame 200 ovalizes) while outer frame 100 is deformed. The length of arm 224 may be increased by increasing the length of the first and/or second section 226, 230 of material. According to some examples, the first section 226 and/or second section 230 of material for each of the plurality of arms 224 may be the same length or different lengths.

While arms 224 are shown with two sections 226, 230 of material separated by a hinge point or bend 228, arms 224 may have any number of sections of material with any number of bends or hinge points. For example, as shown in FIG. 2B, arms 224 may only include the first section 226 of material and no bends. In some examples, arms 224 may have a double bend, such that arms 224 form a “Z-like” shape, or have three bend points, such that arms 224 form a “zig-zag-zig” shape. Additionally or alternatively, the sections of material may have any shape. For example, the first section 226 of material is shown as having a curve. However, the first section 226 and/or second section 230 may be linear, zig-zag, wavy, etc. Thus, arms 224, as shown in FIGS. 2A and 2B, are merely exemplary and are not intended to be limiting.

According to some examples, arms 224 may extend from inner frame 200 rather than outer frame 100 such that all of the available material during manufacturing of outer frame 100 may be used to achieve maximal stiffness while also minimizing the packing diameter.

FIG. 2C is an example inner frame 200C that is connected to outer frame 100 via one or more arms 218a. Although the term “arms” is used, in this embodiment, the “arms” may also be thought of as extended portions of the diamond shaped cells 208 in the first row 210, or simply as arches 218a. Arms 218a may be flexible such that when arms 218a are connected to an outer frame, arms 218a may be curved into a “U” shape, or an arch. Additionally or alternatively, when arms 218a are connected to the outer frame, arms 218a may create any shape or may be bent such that arms 218a include one or more bend points. Arms 218a may be manufactured to have any length. For example, a smaller inner frame 200C being coupled to a large outer frame may include longer arms 218a as compared to a smaller inner frame 200C being coupled to a smaller outer frame. According to some examples, arms 218a may be coupled to the atrial end, or disk, of outer frame. Additionally or alternatively, arms 218a may be coupled to the waist, or central portion, of outer frame.

In all of the embodiments described herein, the various arms 224 (or arms/arches 218a in connection with FIG. 2C) may be formed integrally with the remainder of inner stent 200, or otherwise formed separately and later coupled to the inner stent 200.

Inner frame 200 and outer frame 100 may be coupled to each other prior to and/or during implantation. Coupling arms 224 of inner frame 200 to outer frame 100 may isolate inner frame 200 from outer frame 100. For example, forces exerted on outer frame 100 may be absorbed by arms 224 such that inner frame 200 is not compressed (or not meaningfully or significantly compressed) by the forces exerted on outer frame 100. This may allow inner frame 200 to maintain a round shape while outer frame 100 is deformed by external forces.

The amount of force that arms 224 (e.g. as shown in FIGS. 2A-C) may absorb may be based on the number of arms 224 coupling inner frame 200 to outer frame 100, where the arms 224 are located along the perimeter of atrial end 202, the length of arms 224, the number of bend points in arms 224, etc. For example, elongating the arms 224 may generally increase the amount of force that can be absorbed. The more force absorbed by the arms 224, the less deformation inner frame 200 may experience when a force is being exerted on outer frame 100. According to some examples, a smaller number of arms 224 coupling inner frame 200 to outer frame 100 may increase the isolation of inner frame 200 from outer frame 100. The more isolated inner frame 200 is from outer frame 100, the less deformation inner frame 200 may experience when a force is being exerted on outer frame 100. In examples where the arms 224 are longer, the arms 224 may be able to absorb more force as compared to arms 224 that are shorter. In some examples, arms 224 that include more bends 228 may absorb more force than arms 224 with fewer bends 228. By increasing the amount of force absorbed by arms 224 and/or increasing the isolation of inner frame 200, the more likely it is that inner frame 200 maintains its shape while a force is being exerted on outer frame 100. This may allow for the prosthetic valve leaflets to coapt more efficiently.

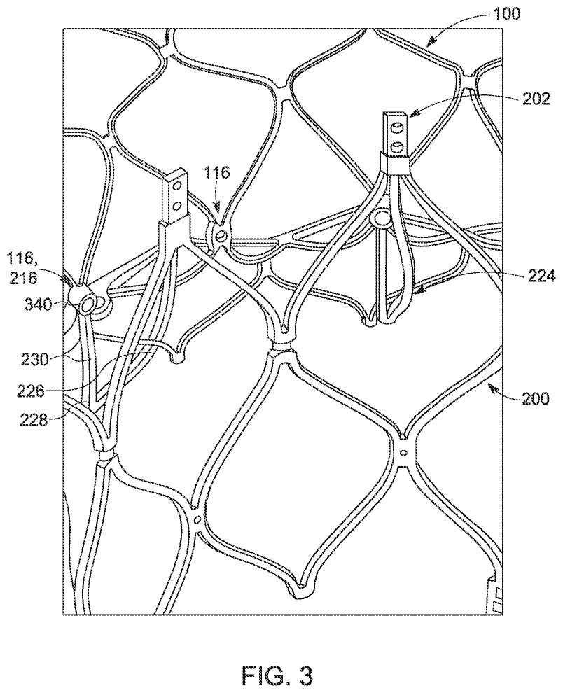

FIG. 3 is a perspective view of the connection between inner frame 200 and outer frame 100. The inner frame 200 may be coupled to outer frame 100 via one or more rivets, sutures, tethers, polymer, an interlocking system, etc. For example, the atrial tip of the second section 230 may include an aperture 216. Aperture 216 of inner frame 200 may be aligned with aperture 116 of outer frame 100 such that inner frame 200 may be coupled to outer frame 100. A rivet may be used to connect apertures 116, 216. According to some examples, apertures 216, 116 may be a connection point for coupling inner frame 200 and outer frame 100 using sutures, tethers, polymer, an interlocking system, etc. Thus, while apertures 116, 216 are shown, they are merely one example of how inner frame 200 and outer frame 100 may be coupled and, therefore, are not intended to be limiting.

In examples where tethers are used to couple inner frame 200 and outer frame 100, the tether may allow inner frame 200 be positioned within outer frame 100 without having a stiff or rigid structure therebetween. The tethers may, in some examples, prevent any compression forces being exerted on outer frame 100 from being exerted on, or transferred to, inner frame 200. For example, the tethers may allow inner frame 200 to float within the central opening of outer frame 100. As the tethers are not a rigid structure, any force exerted on outer frame 100 will be absorbed by outer frame 100 and would not be transferred by the tethers to inner frame 200.

According to some examples, inner frame 200A-C may be coupled to outer frame 100 at the atrial end of outer frame. The atrium typically does not contract as much as the ventricle during a heart cycle such that the ventricular end of outer frame 100 may experience more deformation than the atrial end of outer frame 100 during a heart cycle. In some examples, the atrial end of outer frame 100 does not substantially deform. By connecting inner frame 200A-C to the atrial end of outer frame 100, less force may be exerted on the atrial end of outer frame 100 and, therefore, less force may be exerted on inner frame 200A-C. In examples where less force is exerted on the atrial end of outer frame 100, arms 224 may have less force and/or deformation to absorb to prevent deformation of inner frame 200A-C. According to some examples, inner frame 200A-C may have fewer number of arms 224 when inner frame 200 is coupled to the atrial end of outer frame 100 compared to when inner frame 200A-C is coupled to the ventricular end of outer frame 100. Fewer arms 224 may be needed to absorb the force exerted on outer frame 100 when inner frame 200A-C is coupled to the atrial end of outer frame 100 as less force is exerted on the atrial end of outer frame 100 as compared to the ventricular end of outer frame 100.

According to some examples, the number of cells 208 around the circumference of the inner frame 200 may be a factor, or multiple (i.e. whole number multiple), of the number of cells 108 around the circumference of the outer frame 100. For example, if the circumference of the inner frame 200 has a total of nine cells 208, the circumference of the outer frame 200 may have a total of nine, eighteen (18), twenty-seven (27), etc. cells 108. By having the number of cells 108, 208 be a factor of each other, there may be symmetry between the connection points of the inner frame 200 and outer frame 100. According to some examples, a nine cell 208 geometry with three (3) connection points may maintain symmetry between the inner frame 200 and outer frame 100. In other examples, an eight (8) cell geometry with two (2), four (4), or eight (8) connection points may maintain symmetry between the inner frame 200 and outer frame 100. Symmetry between the connection points of inner frame 200 and outer frame 100 may balance the load in the sheath of the delivery device when implanting the replacement valve.

According to some examples, however, the connection points may be asymmetrical. The asymmetrical connection points may compensate for the asymmetrical, or “D-shape,” of a valve annulus. For example, there may be more connection points on a short axis as there is likely to be more pressure, or a greater amount of force, being exerted on the outer frame 100 along the short axis. By having more connection points and/or arms 224 along the short axis, the arms 224 may absorb the force thereby preventing the force from being transferred to the inner frame 200. The short axis may be defined by the catheter once the catheter is inserted into the heart. By defining the short axis via the catheter, that axis can be used to define the orientation of the replacement valve when loading the replacement valve in the catheter during implantation. This may ensure that the axis, or portions, with the greater number of connection points and arms 224 is in the correct orientation.

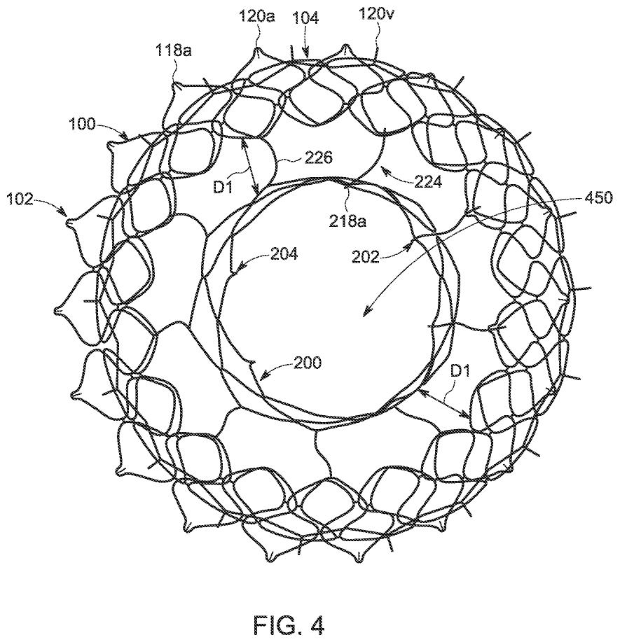

FIG. 4 illustrates an example of a replacement heart valve having an inner frame, an outer frame, and arms extending therebetween. According to some examples, the inner frame 200 may have an outer diameter of about 27 mm and the central portion 106 of outer frame 100 may have an outer diameter of about 40 mm. According to some examples, the central portion 106 of outer frame 100 may have an outer diameter between about 32 mm and about 48 mm. However, the central portion 106 of outer frame 100 may have an outer diameter smaller than 32 mm or greater than 48 mm and, therefore, the range of 32-48 mm is merely exemplary and is not intended to be limiting. The diameter of the central portion 106 the outer frame 100 may be determined based on the length of arms 224. For example, a larger diameter of central portion 106 may cause a physician to choose an inner frame 200 with longer arms 224 as compared to an outer frame 100 with a smaller diameter central portion 106.

As shown, inner frame 200 is located within the central opening 450 of outer frame 100. Arms 224 of inner frame 200 extend a distance outward from inner frame 200 and are coupled to outer frame 100. The arms 224 define a distance “D1” between outer frame 100 and inner frame 200. The distance “D1” corresponds to the distance arms 224 may be compressed before an external force exerted on outer frame 100 will be exerted (or meaningfully exerted) on inner frame 200. The amount of force that may be absorbed by arms 224 may correspond to the amount of potential energy that can be absorbed by arms 224. For example, the arms 224 may act similar to a spring such that each arm 224 may have a compression constant, or spring constant. The compression constant may be determined based on the type of material the arm 224 is made of, the length of the arm 224, the number of bends in the arm, the thickness of the material that the arms 224 are made of, the width of the arm 224, etc. For example, the thickness and/or width of arms 224 may be varied along the length of the arm 224 to as a way to distribute the force along the arm 224. In some examples, the thickness and/or width of the arms 224 may be varied along the length of the arm 224 to increase the amount of force that can be absorbed by the arm. The amount of energy, and therefore force, that can be absorbed by the arm 224 may be based on the distance “D1” that the arm 224 can be compressed. The greater the compression constant and/or distance the arm 224 can be compressed, the greater the amount of energy, and therefore force, each arm 224 can absorb.

Using arms 224 to isolate inner frame 200 from forces exerted on outer frame 100 may allow for the outer frame 100 to expand to a larger diameter while maximizing the material of the outer frame and allowing for various sized inner frames. For example, the outer frame 100 may have a closed cell shape such that none of the material for the outer frame 100 is used to connect the outer frame 100 to inner frame 200. This may allow for the outer frame 100 to maintain the necessary stiffness while still being able to be coupled to an inner frame 200.

In some examples, the physician may subsequently be able to insert a second inner frame within the inner frame 200. This may allow the physician to replace the inner frame 200 independent from the outer frame 100 due to, for example, degraded leaflets.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A replacement cardiac valve, comprising:
an outer frame configured to expand from a collapsed configuration to an expanded configuration;
an inner frame configured to expand from a collapsed configuration to an expanded configuration; and
one or more arms extending between the inner frame and the outer frame, wherein the one or more arms are configured to isolate the inner frame such that a force exerted on the outer frame is absorbed by the outer frame and the one or more arms.

2. The replacement cardiac valve of claim 1, wherein the one or more arms extend from the inner frame and are coupled to the outer frame.

3. The replacement cardiac valve of claim 2, wherein the outer frames includes a first plurality of diamond shaped cells and the inner frame includes a second plurality of diamond shaped cells such that each of the one or more arms extend from an apex of the second plurality of cells to a connection point between adjacent cells of the first plurality of cells.

4. The replacement cardiac valve of claim 2, wherein the each of the one or more arms are coupled to the outer frame by a rivet, a suture, a polymer, or an interlocking system.

5. The replacement cardiac valve of claim 1, wherein the one or more arms has at least one hinge point.

6. The replacement cardiac valve of claim 5, wherein each of the at least one hinge point corresponds to a change in direction of the arms.

7. The replacement cardiac valve of claim 1, wherein the one or more arms is a tether.

8. The replacement cardiac valve of claim 1, wherein a circumference of the outer frame includes a first number of cells and a circumference of the inner frame includes a second number of cells, the first number of cells being a multiple of the second number of cells.

9. A replacement cardiac valve, comprising:
an outer frame having a first plurality of diamond shaped cells, the outer frame configured to expand from a collapsed configuration to an expanded configuration;
an inner frame having a second plurality of diamond shaped cells, the inner frame configured to expand from a collapsed configuration to an expanded configuration; and
one or more arms extending from the inner frame to the outer frame, wherein each of the one or more arms extends from an apex of the second plurality of diamond shapes cells and is coupled to an apex of the first plurality of diamond shaped cells.

10. The replacement cardiac valve of claim 9, wherein the one or more arms are configured to isolate the inner frame such that a force exerted on the outer frame is absorbed by the outer frame and the one or more arms.

11. The replacement cardiac valve of claim 9, wherein the each of the one or more arms are coupled to the outer frame by a rivet, a suture, a polymer, or an interlocking system.

12. The replacement cardiac valve of claim 9, wherein the one or more arms has at least one bend point.

13. The replacement cardiac valve of claim 12, wherein each of the at least one bend point corresponds to a change in direction of the arms.

14. The replacement cardiac valve of claim 9, wherein the one or more arms is a tether.

15. A replacement cardiac valve, comprising:
an outer frame configured to expand from a collapsed configuration to an expanded configuration;
an inner frame configured to expand from a collapsed configuration to an expanded configuration; and
one or more arms extending from the inner frame and coupled to the outer frame,
wherein each of the one or more arms has a bend point corresponding to a change in direction of each of the one or more anchoring mechanisms, and
wherein the one or more arms are configured to isolate the inner frame such that a force exerted on the outer frame is absorbed by the outer frame and the one or more arms.

16. The replacement cardiac valve of claim 15, wherein the each of the one or more arms are coupled to the outer frame by a rivet, a suture, a polymer, or an interlocking system.

17. The replacement cardiac valve of claim 15, wherein the outer frame has a first plurality of diamond shaped cells and the outer frame has a second plurality of diamond shaped cells.

18. The replacement cardiac valve of claim 17, wherein the first plurality of diamond shaped cells is a multiple of the second plurality of diamond shaped cells.

19. The replacement cardiac valve of claim 17, wherein each of the one or more arms extends from an apex of the second plurality of diamond shaped cells.

20. The replacement cardiac valve of claim 17, wherein each of the one or more arms is coupled to the outer frame at an apex where adjacent cells of the first plurality of diamond shaped cells adjoin.

21. A replacement cardiac valve, comprising:
an outer frame having a first plurality of diamond shaped cells, the outer frame configured to expand from a collapsed configuration to an expanded configuration;
an inner frame having a second plurality of diamond shaped cells, the inner frame configured to expand from a collapsed configuration to an expanded configuration; and
one or more arms extending an apex of the second plurality of cells to a connection point between adjacent cells of the first plurality of cells,
wherein each of the one or more arms includes one or more portions, a first portion of each of the one or more arms extending in a first direction and a second portion of each of the one or more arms extending in a second direction opposite the first direction,
wherein each of the one or more arms has a bend point between the first portion and the second portion, and
wherein the one or more arms are configured to isolate the inner frame such that a force exerted on the outer frame is absorbed by the outer frame and the one or more arms.
